(19) United States
(12) Patent Application Publication
(10) Pub. No.: US 2021/0378900 A1
(43) Pub. Date: Dec. 9, 2021

(54) Chair mounted patient isolation container

(71) Applicant: Ernest Jason Washburn, Opelika, AL (US)

(72) Inventor: Ernest Jason Washburn, Opelika, AL (US)

(21) Appl. No.: 16/846,069

(22) Filed: Jun. 3, 2020

(51) Int. Cl. A61G 15/10; A61F 9/04

(52) U.S. Cl. CPC A61G 15/10; A61F 9/045

(57) ABSTRACT

A chair mounted patient isolation container that increases the effective containment of aerosols is disclosed. In one embodiment, the isolation container includes a plurality of panels, a plurality of tabs and slots for connecting said panels, clinical access ports to allow for maximum clinical range of motion for the dental service provider, and ports for connection of external clinical devices such as external scavenging suction devices for further elimination of aerosol contamination. One embodiment of the container comprises means for mounting the container to a dental procedure chair, such that the device generally encloses the patient's head and upper torso when the patient is positioned on the dental procedure chair.

### FIELD OF THE INVENTION

The present disclosure relates generally to devices for reducing the spread of infectious diseases during various medical and dental procedures. More particularly, the present disclosure relates to a protective barrier mounted to dental chairs to increase containment of and reduce the exchange and spread of germs and other potentially infectious biologic aerosols that often exist during dental procedures.

### BACKGROUND OF THE INVENTION

During common dental treatments and procedures there is an increased risk of infection due to the dispersion of bodily fluids of patients and bacteria into the environment from the oral cavity and upper respiratory tract. Aerosols containing saliva, blood, dust, bacteria, etc. created during dental procedures present a significant risk for infection in dental clinics. Moreover, according to CDC and other health officials, these aerosols could contain infectious viruses. The onset of the recent COVID-19 pandemic has only made more acute the need to address the risks of infection presented by surgical and other oral procedures.

Several current measures exist in the prior art to mitigate the spread of aerosols in the dental environment, however, there remains a high risk of potential infection and a need for more effective solutions. For example, high volume intra-oral suctions, rubber dam isolation, and open space extra-oral vacuums have been found to help reduce the release of aerosols during the procedure; however, it will be appreciated that utilization of these measures only achieves limited containment of aerosols, presenting a significant risk that unwanted air-born based substances will escape, disseminate and contaminate the dental environment. Also, these aerosol reduction devices/methods do not, and are not designed to, isolate a patient's emissions from coughing or sneezing during a procedure, thus creating additional non-procedure-caused aerosols into the clinical room environment.

Other measures, such as an intubation box, are utilized in surgical procedures in which the patient is positioned supine for receiving certain endotracheal treatments. However, such measures are not effective in the dental environment. Measures such as an intubation box are limited in that the intubation box must be placed on a horizontal fixed surface and when a patient is lying in a generally supine position. Intubation boxes provide medical service providers with limited range of motion and visibility and do not provide for the use of suctioning devices. Dental procedures are rarely conducted while a patient is lying in flat parallel position and require the ability to adjust the position of the patient, including while the procedure, is in progress. Dental service providers have a need for “scavenging” airborne-based materials through the use of suctioning. Thus, there is a need for enhanced protective measures that facilitate greater access and range of motion for the service provider to a patient while maintaining maximum protection and containment to prevent the spread of infectious diseases in the clinical setting.

The Chair Mounted Patient Isolation Container, as described herein, effectively isolates and contains not only procedural produced aerosols but also those that are patient produced.

### SUMMARY OF THE INVENTION

The present disclosure describes a chair mounted patient isolation container that increases the effective containment of aerosols and thereby reduces the risk of the spread and exchange of bacteria, viruses and other infectious disease during dental procedures by providing a translucent barrier between the patient and the dental service provider performing the dental procedure. The chair mounted patient isolation container is made from a transparent material thus providing the dental service provider with unobstructed visibility during dental procedures. The said container comprises specially designed, sized and positioned clinical access ports to allow for maximum clinical range of motion for the dental service provider while limiting exposure to and release of potentially infectious aerosols. The transparent structure and ports provide the dental service provider complete or near-complete accessibility, from all angles, relative to the patient, without requiring precise patient positioning. The said container further comprises ports for connection of external clinical devices such as external scavenging suction devices for further elimination of aerosol contamination. The said container further comprises means for mounting to the dental procedure chair, such that the device generally encloses the patient's head and upper torso when the patient is positioned on the dental procedure chair. A surrounding barrier between the patient and dental services provider is thereby provided. The said container further comprises component panels releasably affixed to one another in sealed fashion that facilitates easy assembly and disassembly. This feature promotes cleaning and sanitizing between patient procedures, as well as replacement of parts in the event of damage or wear.

### BRIEF DESCRIPTION OF THE DRAWINGS

Further features and advantages of the invention can be ascertained from the following detailed description that is provided in connection with the drawings described below:

FIG. 1 shows an embodiment of the chair mounted patient isolation container.

FIG. 2 shows a rear view of an embodiment of the chair mounted patient isolation container.

FIG. 3 shows a bottom view of an embodiment of the chair mounted patient isolation container.

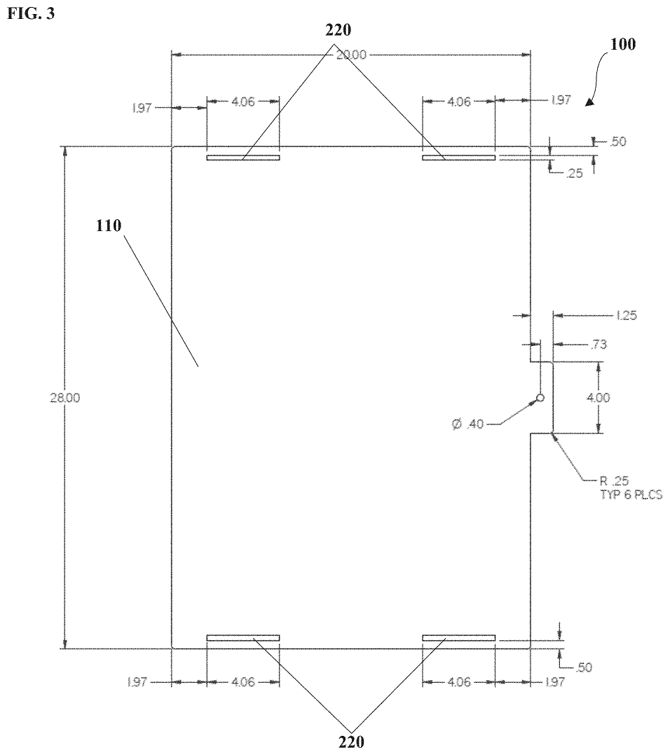

FIG. 4 shows a side view of an embodiment of the chair mounted patient isolation container.

FIG. 5 shows an embodiment of the quick release pins.

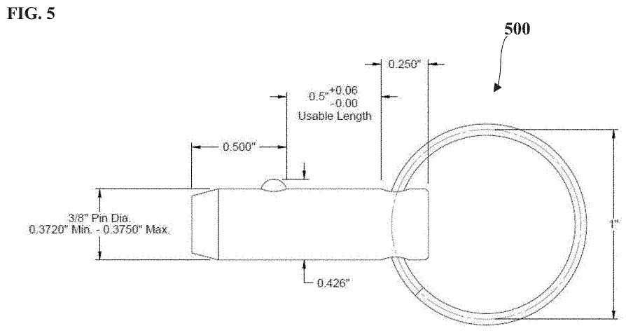

FIG. 6 shows an embodiment of the chair mounted patient isolation container connected to the back portion of a dental procedure chair.

FIG. 7 shows an embodiment of the chair mounted patient isolation container with scavenging suction devices attached.

### DETAILED DESCRIPTION OF THE INVENTION

### A. Definitions

Unless otherwise defined, all terms (including technical and scientific terms) used herein have the same meaning as, commonly understood by one of ordinary, skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity.

The terms “about” and “approximately” shall generally, mean an acceptable degree of error or variation for the quantity measured given the nature or precision of the measurements. Typical, exemplary degrees of error or variation are within 20 percent (%), preferably within 10% and more preferably within 5% of a given value or range of values. Numerical quantities given in this description are approximate unless stated otherwise, meaning that the term “about” or “approximately” can be inferred when not expressly stated.

It will be understood that when a feature or element is referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments.

The terminology used herein is for the purpose of describing particular embodiments only and is not intended to be limiting. As used herein, the singular forms “a”, “an” and “the” are intended to include the plural forms as well (i.e., at least one of whatever the article modifies), unless the context clearly indicates otherwise.

Spatially relative terms, such as “under”, “below”, “lower”, “over”, “upper” and the like, may be used herein for ease of description to describe one element or feature's relationship to another when the apparatus is mounted to a medical or dental chair.

The terms “first”, “second”, and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

The term “consisting essentially of” means that, in addition to the recited elements, what is claimed may also contain other elements (steps, structures, ingredients, components, etc.) that do not adversely affect the operability of what is claimed for its intended purpose as stated in this disclosure. This term excludes such other elements that adversely affect the operability of what is claimed for its intended purpose as stated in this disclosure, even if such other elements might enhance the operability of what is claimed for some other purpose.

### B. Chair Mounted Patient Isolation Container

A chair mounted patient isolation container (“container”) for reducing the spread of infectious diseases during various medical and dental procedures is herein disclosed. In a general embodiment of the disclosed invention herein, the container 100 comprises a plurality of transparent panels releasably attached to one another to form a generally rectangular structure that encapsulates and provides unobstructed visibility to a patient receiving dental and oral treatments. One of said panels is connected to the dental procedure chair by an attachment means to be described further below. In a preferred embodiment, there are five panels consisting essentially of a front 140, rear 130, top 110, and two side panels 120. Each said panel comprises polycarbonate or other translucent and rigid material capable of being assembled into a rigid structure. In the preferred embodiment, said panels are rectangular in shape and, upon assembly, comprise a generally rectangular containment structure that is open on one side to encapsulate the upper portion and head of the patient upon fixation to the chair or table upon which the patient is positioned for treatment. It will be appreciated by those skilled in the art that while the rectangular shape promotes relatively simple assembly and disassembly, while allowing a full range of access by the dental team to the patient through clinical access ports 160 to be described further herein, other containment shapes are conceivable.

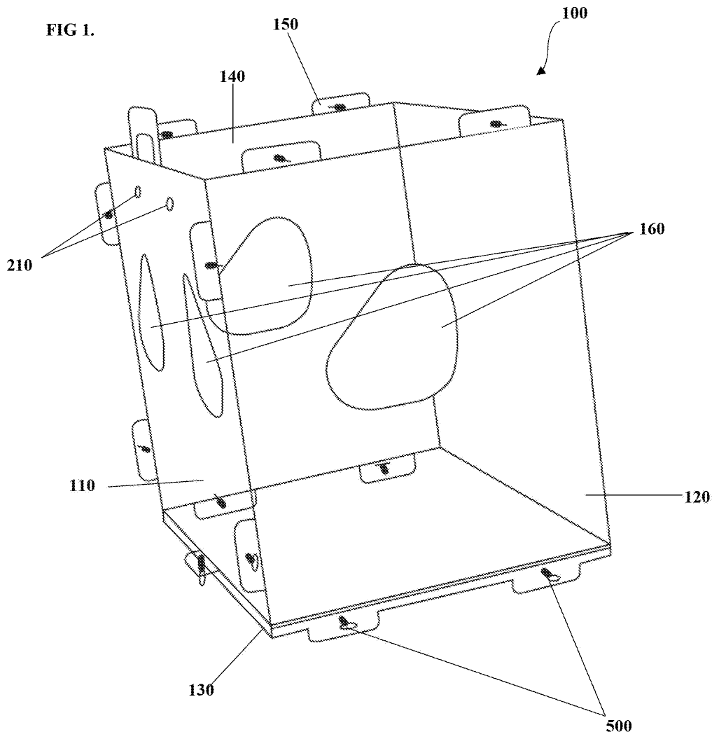

As shown in FIG. 1, said panels of the mounted patient isolation container comprise tabs 150 and slots 220 for ease of assembly and structural integrity, located near the periphery of each said panel. The said tabs 150 are inserted into said slots 220 as part of the assembly process. The assembly is completed with the use of quick release pins 500 which ensure the panels are secure while in use. As shown in FIG. 1, the tabs and quick release pins 500 allow the said container 100 to be quickly and easily assembled after the patient is positioned in the dental procedure chair. Likewise, the invention affords relatively simple disassembly permitting prompt disinfection and sterilization.

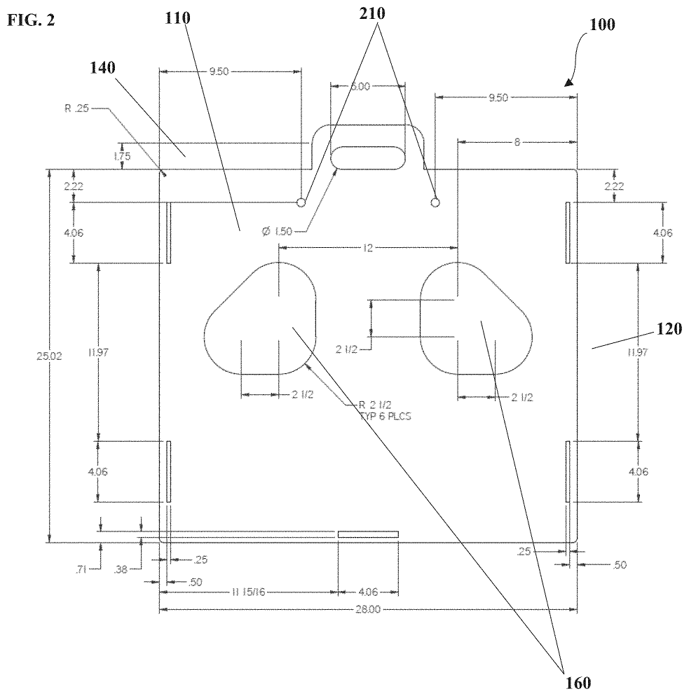

Referring now to FIGS. 1, 2, and 4, the claimed invention further comprises a plurality of clinical access ports 160 to allow the dental services provider(s) to access the patient while maintaining a barrier between themselves and the patient. As shown in FIG. 2, the top panel 110 and each of the side panels 120 hosts at least one of the said clinical access ports 160 which are positioned, sized and shaped to allow for the dental service provider and at least one other assistant to have free range of motion and patient access to perform dental procedures while reducing exposure to aerosol contaminants.

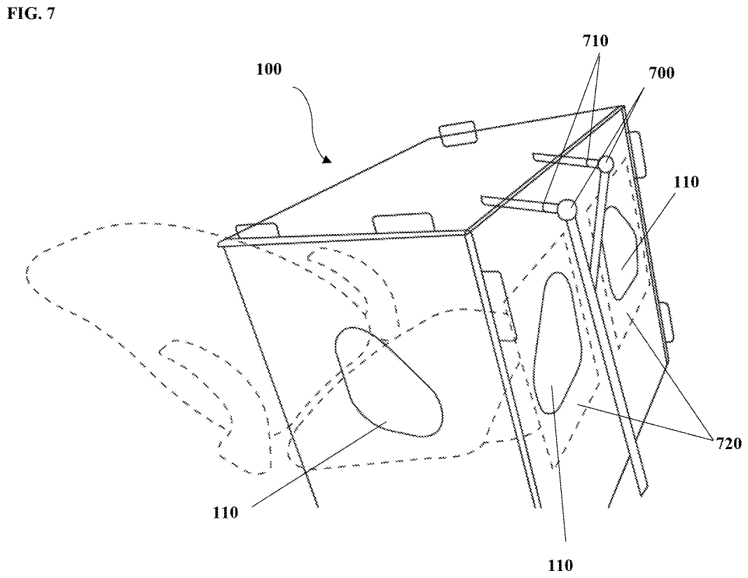

In one embodiment, a pair of clinical access ports 160 are positioned in said top panel 110 and a single access port is positioned in each of said side panels 120. In said embodiment, the clinical access ports 160 on each side panel 120 are positioned generally in the center of each respective side panel 120. The clinical access ports 160 on the top panel 110 are generally positioned equal distance apart in the center of the top panel 110 and equal distance from each edge of the respective side panel 120. The clinical access port 160 are generally sized for the circumference of an average adult arm. As shown in FIG. 2, in an exemplary embodiment of the claimed invention, the clinical access ports 160 are generally triangular in shape with rounded corners with an opening surface area of about 35 square inches each (225.806 square centimeters). As shown in FIG. 7, the clinical access ports 160 may be sealed by appropriately shaped coverings 720 when not in use.

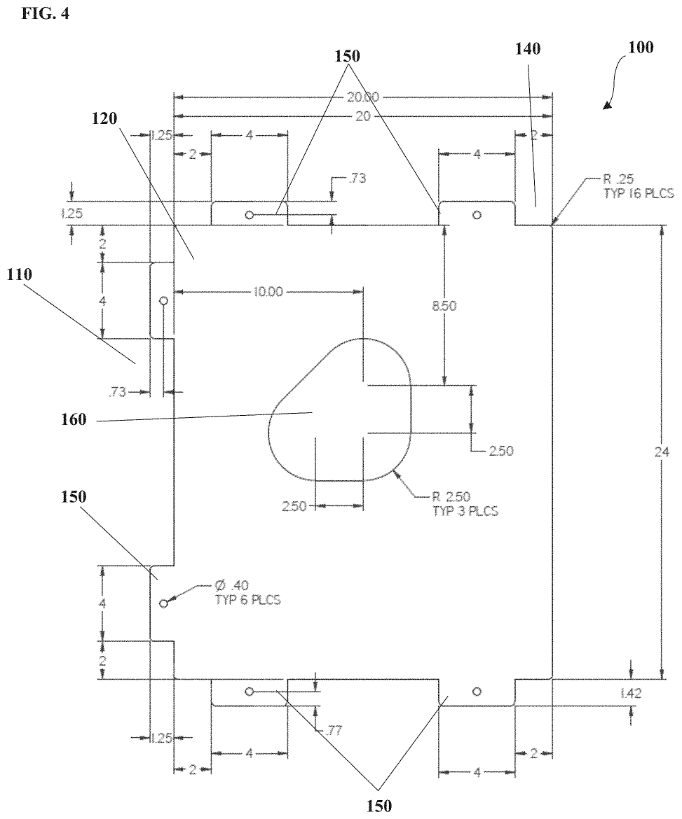

As shown in FIG. 4, in an exemplary embodiment, at least one clinical access port 160 is located on each side panel 120 (about at patient's ears) of the isolation container with the center of the clinical access port positioned about 8.75 inches (222.25 millimeters) from the edge of the top panel 110 of the isolation container and about 9.75 inches (247.65 millimeters) from the edge of the front panel 140 of the isolation container. As shown in FIG. 2, said pair of clinical ports is located on the top panel 110 (at crown of patient's head) of the isolation container, the center of each of which is about 6.0 inches (152.4 millimeters) from the edge of the respective side panels 120 of the container and about 10 inches (254 millimeters) from the edge of the front panel 140 of the container.

As shown in FIG. 2, in a preferred embodiment, at least one of the said panels 110, 120, 130, 140 of the said container 100 comprises a plurality of scavenging access ports 210 sized for receiving external scavenging suction tubes to aid with aerosol elimination. As shown in FIG. 2, the scavenging device access ports 210 are generally positioned above the clinical access ports 160 equal distance apart and equal distance from the edge of each respective side panel 120. As shown in FIG. 7, in the preferred embodiment, the scavenging suction tubes 700 are inserted into the scavenging device access ports 210 and secured to the ports using rubber o-rings 710 and sealed by the tube itself filling the port opening.

In one embodiment of the claimed invention, shown in FIG. 2, the scavenging device access ports 210 are positioned on at least the top panel 110 of the isolation container and allow for the insertion of scavenging suction tubes for an increased vacuum environment that promotes the further containment of aerosols. In an, exemplary embodiment of the claimed invention the scavenging device access ports 210 have a diameter of about ⅝ inches (14.28 millimeters) and are generally round in shape. As shown in FIG. 2, the center of the scavenging device access ports are positioned on the top panel 110 about 2.22 inches (56.388 millimeters) from the edge of the front panel 140 of the isolation container and about 9.50 inches (241.3 millimeters) from the edge of each side panel 120 respectively. The scavenging device access ports 210 may be sealed by appropriately shaped coverings when not in use.

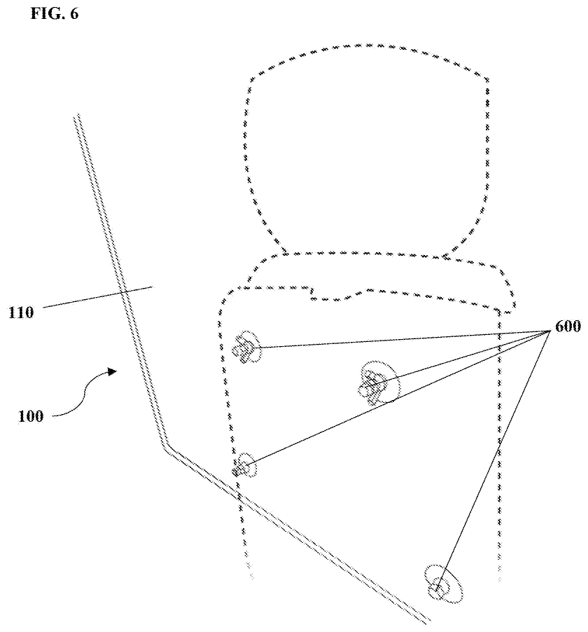

The said container 100 is connected to the back section of a standard dental procedure chair and secured using an attachment means. In a preferred embodiment of the claimed invention, the attachment means comprises a plurality of apertures located within one of said panels and threaded rods and securing hardware 600. The threaded rods extending from the patient chair and securing hardware 600 allow for the patient isolation container 100 to be easily attached to the back portion of a standard dental procedure chair, as shown in FIG. 6. The attachment hardware also allows for easy disassembly from the back section of said chair. In an exemplary embodiment of the claimed invention the attachment means comprises at least four (4) threaded rods and a plurality of washers and nuts. As shown in FIG. 6, many dental procedure chairs comprise a rigid metal bracing that will allow for bolts to be attached through the metal bracing and through the bottom panel of the container. Once reassembled, the dental procedure chair now has at least four (4) threaded rods extruding from the back to allow for the attachment portion of the said container to be connected. The attachment portion is then slid into place over the extruding threaded rods after which washers and nuts (or other appropriate attachment hardware) are fastened to securely hold the attachment portion of the container to the back of said chair. It will be appreciated that when so connected, the said container 100 surrounds a patient's head while said patient is lying, on the dental procedure chair thus providing a barrier between the patient and the dental service provider while allowing the dental service provider 360-degree view of and access to a patient in a variety of positions. Because the container 100 is connected to the back section of the dental procedure chair, said container 100 moves with the chair when the chair height and position are adjusted during procedures thus providing optimal patient and provider comfort.

It will be appreciated by those skilled in the arts that the said panels of the above described invention can be made in different sizes to provide different enclosure capacity, appropriately sized for patients ranging from small children to large adults.

### C. Conclusions

It is to be understood that any given elements of the disclosed embodiments of the invention may be embodied in a single structure, a single step, a single substance, or the like. Similarly, a given element of the disclosed embodiment may be embodied in multiple structures, steps, substances, or the like.

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but, as mentioned above, it is to be understood that the teachings of the present disclosure are capable of use in various other combinations, modifications, and environments and are capable of changes or modifications within the scope of the teachings as expresses herein, commensurate with the skill and/or knowledge of a person having ordinary skill in the relevant art. The embodiments described hereinabove are further intended to explain certain best modes known of practicing the processes, machines, manufactures, compositions of matter, and other teachings of the present disclosure and to enable others skilled in the art to utilize the teaching of the present disclosure in such, or other, embodiments and with the various modifications required by the particular applications or uses. Accordingly, the processes, machines, manufactures, compositions of matter, and other teachings of the present disclosure are not intended to limit the exact embodiments and examples disclosed herein. Any section headings herein are provided only for consistency with the suggestions of 37 C.F.R. § 1.77 or otherwise to provide organizational queues. These headings shall not limit or characterize the invention(s) set forth herein.

What is claimed is:

1. A chair mounted patient isolation container providing a barrier between medical service providers and patients comprising:
a plurality of panels; and
a plurality of clinical access ports positioned to provide the medical or dental service provider full range of access to said patient; and
a plurality of scavenging device access ports; and
a plurality of tabs and slots for connecting said plurality of panels.

2. The panels of claim 1 comprising a front, rear, top, and two side panels, said panels being releasably attached.

3. The panels of claim 1 wherein said panels comprise transparent material.

4. The panels of claim 1 wherein said panels comprise transparent polycarbonate material.

5. The clinical access ports of claim 1 wherein said clinical access ports comprise a generally triangular shape having rounded corners.

6. The clinical access ports of claim 1 wherein said clinical access ports have an opening circumference of about 35 square inches.

7. The clinical access ports of claim 1 wherein at least one said clinical access port is positioned on the top and each of the two side panels.

8. The clinical access ports of claim 7 wherein the said clinical access port positioned on each side panel is positioned generally in the center of each the said respective side panels.

9. The clinical access ports of claim 8 wherein the said clinical access ports are positioned about 8.75 inches (222.25 millimeters) from the edge of the top panel of the isolation container and about 9.75 inches (247.65 millimeters) from the edge of the front panel of the isolation container.

10. The clinical access ports of claim 1 wherein at least two clinical access ports are positioned on the top panel.

11. The clinical access ports of claim 10 wherein said clinical access ports are positioned equal distance apart in the center of the top panel and equal distance from each edge of the respective side panel.

12. The clinical access ports of claim 10 wherein the center of the said clinical access ports are positioned about 6.0 inches (152.4 millimeters) from the edge of the respective side panels of the isolation container and about 10 inches (254 millimeters) from the edge of the front panel of the isolation container.

13. The scavenging device access ports of claim 1 wherein at least one said scavenging device access ports is positioned on the top panel of the patient isolation container.

14. The scavenging device access ports of claim 1 wherein at least two said scavenging device access ports are positioned on the top panel equal distance apart in the center of the top panel and equal distance from each edge of the respective side panel.

15. The scavenging device access ports of claim 13 wherein the center of the said scavenging device access ports are positioned about 2.22 inches (56.388 millimeters) from the edge of the front panel and about 9.5 inches (241.3 millimeters) from the edge of each side panel.

16. The isolation container of claim 1 wherein the said plurality of tabs are secured by a plurality of quick release pins.

17. The isolation container of claim 1, further comprising a means of attaching the said isolation container to a dental procedure chair.

18. The attachment means of claim 17, wherein said attachment means comprises at least four threaded rods.

19. The attachment means of claim 18, further comprising a plurality of washers and nuts

20. The attachment means of claim 19, further comprising a plurality of apertures located within one of said panels of the isolation container.
